(19) United States
(12) Patent Application Publication
(10) Pub. No.: US 2019/0002825 A1
(43) Pub. Date: Jan. 3, 2019

(54) Method for Inducing Targeted Differentiation of Human Stem Cells Toward Hepatic Cells

(71) Applicant: TRANSCEND CYTOTHERAPY CO., LTD, Jiangsu (CN)

(72) Inventors: Pei-Lin ZHANG, Jiangsu (CN); Li-Xin CHEN, Jiangsu (CN)

(21) Appl. No.: 15/764,508

(22) PCT Filed: Nov. 29, 2016
(86) PCT No.: PCT/CN2016/107647

§ 371 Date: Sep. 10, 2018

(30) Foreign Application Priority Data
Sep. 30, 2015 (CN) 201510640312.3

(51) Int. Cl. C12N 5/071; C12N 5/0775; C12N 5/0735; C12N 5/074

(52) U.S. Cl. CPC C12N 5/067; C12N 5/0663; C12N 5/0606; C12N 5/0696; C12N 2501/237; C12N 2501/39; C12N 2501/385; C12N 2501/70; C12N 2501/12; C12N 2506/02

(57) ABSTRACT

The present invention relates to a novel method for multi-target-directed inducing direct differentiation of human stem cells, such as human embryonic stem cells (ES cells) or induced pluripotent stem cells (iPS cells), into hepatocytes using combinations of small molecules. The present invention discloses a medium and a culturing method for inducing directed differentiation of human stem cells into hepatocytes directly. In the method of the present invention, no exogenous genes are need to be introduced into stem cells, no stepwise induction is needed, various cell growth factors are not needed, and directed differentiation of human stem cells into hepatocytes directly can be achieved using only small chemical molecules. The differentiated human hepatocytes obtained have the typical characteristics of human hepatocytes, the differentiated liver precursor cells can be passaged for a long period of time, and the differentiated liver mature cells can be passaged for a limited number of times. Moreover, the method uses a conventional culturing procedure with simple operation, low cost, safety and stability.

### TECHNICAL FIELD

The present invention falls within the fields of biology and medicine; more specifically, the present invention relates to a novel method for multi-target-directed inducing direct differentiation of human stem cells, such as human embryonic stem cells or induced pluripotent stem cells, into hepatocytes using only combinations of small molecules, and to a dedicated direct differentiation medium used in the novel method.

### BACKGROUND ART

According to the statistics of the World Health Organization, millions of people die of liver diseases each year in the world. China is a country with considerable people suffering from liver diseases. There are 140 million people with HBV and HCV alone, accounting for 28% of the world's total. Acute and chronic liver failures caused by various causes are dangerous in condition, poor in prognosis and have high mortality (70%-80%). Hepatocyte transplantation and bioartificial liver replacement therapy can not only treat liver failures, but also treat genetic metabolic liver diseases and neuropsychiatric disorders caused by elevated blood ammonia due to liver dysfunction; and hepatocyte transplantation can also promote endogenous liver regeneration in patients with acute liver failure. However, in addition to hepatocyte transplantation and bioartificial liver construction, liver toxicity testing of new drugs, researches for liver diseases and the like require a large number of qualified human hepatocytes; but the lack of liver resources is a global problem. Therefore, by virtue of the multi-lineage differentiation potential of human embryonic stem cells (ES cells) and induced pluripotent stem cells (iPS cells), directed induction of same to differentiate into hepatocytes has become one of global hotspots for obtaining sources of hepatocytes. Moreover, iPS cells break the ethical restrictions, so it has received more attention from researchers.

Human iPS cells are induced pluripotent stem cells obtained by introducing 4 genes (Oct4, Sox2, Klf4 and c-Myc, or Oct4, Sox2, Nanog and Lin28) into somatic cells (Takahashi K. Cell 2007; 131: 861-872; and Yu J, et al., Science 2007; 318: 1917-1920). iPS cells have similar properties to ES cells and can differentiate into three germ layers, that is the inner, middle and outer germ layers under specific induction conditions. By virtue of the ability of iPS cells to expand indefinitely in vitro and their multi-lineage differentiation potential, sufficient sources of hepatocytes can be obtained by directed induction of iPS cells to differentiate into hepatocytes. In case that somatic cells can be obtained from a patient and used to establish an iPS cell line with the same genetic background as the patient, and the iPS cell line is induced to differentiate into hepatocytes required by the patient, and the hepatocytes are used in hepatocyte transplantation therapy for the patient, personalized hepatocytes obtained by the method can avoid or reduce immune rejection caused by allogeneic cell transplantation to the utmost extent.

ES/iPS cells have been shown to differentiate into functional hepatocytes in vitro as early as 2007 (Cai J, et al., Hepatology 2007, 45(5): 1229-1239). With the in-depth and extensive development of researches, more, and more effective induction and differentiation methods have emerged. Currently, the most common differentiation method is to induce differentiation of pluripotent stem cells step by step with growth factors as follows: firstly, pluripotent stem cells differentiate and develop into anterior definitive endoderm cells by Nodal signaling and FGF signaling; then, the anterior definitive endoderm cells differentiate into hepatic cells under the effect of growth factors BMP4 and FGF; and finally, hepatocyte growth factor (HGF) and oncostatin M (OSM) are used to promote differentiated hepatocytes to further Mature (Agarwal S, et al., Stem Cells 2008, 26(5): 1117-1127; Hay D C, et al., PNAS 2008, 105(34): 12301-12306; Song Z, et al., Cell Res 2009, 19(11): 1233-1242; Touboul T, et al., Hepatology 2010, 51(5): 1754-1765; Duan Y, et al., Stem Cells 2010, 28(4): 674-686; Si-Tayeb K, et al., Hepatology 2010, 51(1): 297-305; Sullivan G J, et al., Hepatology 2010, 51(1): 329-335; Liu H, et al., Sci Trans Med 2011, 3(82): 82ra39; and Kajiwara M, et al., PNAS 2012, 109(31): 12538-43, 2012). Other methods include induction using human MSCs (Mesenchymal Stem Cells) as feeder cells (Mallanna S K, et al., Curr Protoc Stem Cell Biol 2013. 26: Unit 1G4), three-dimensional culture (Mobarra N, et al., Int J Hematol Oncol Stem Cell Res 2014, 8(4): 20-9), introduction of exogenous genes (Takayama K, et al., PLoS One 2011, 6(7): e21780; Takayama K, et al., Mol Ther 2012, 20(1): 127-37; and Takayama K, et al., Biomaterials 2013. 34(7): 1781-9) or the like. Although significant advances have been made in the researches of directed induction of human ES/iPS cells to differentiate into hepatocytes, the current findings still have such defects and problems as: 1. for most differentiation methods, the culturing conditions for ES/iPS cells still include culturing the cells in layers of mouse feeder cells or the culture solution contains animal-derived components, so differentiated hepatocytes obtained by such methods may be difficult to apply clinically due to the possible presence of unknown animal pathogens; 2. there are too many induction and differentiation steps, and there are too many cell growth factors to be used, so it is difficult to control the stage of differentiation and the final quality, which also leads directly to the high cost of differentiation and difficulty in practical use; 3. most differentiation methods are not efficient in differentiation, and the differentiated hepatocytes need to be subjected to extra purification steps due to their low purity, which increases the production cost, reduces the cell viability and biological activity and causes difficulty in meeting the requirements of clinical application; 4. the mature hepatocyte surface marker ASGPR is absent or low in content (Takayama K, et al., J Hepatol 2012, 57(3): 628-36); 5. functions of the differentiated hepatocytes are compromised, in particular P450 metabolic enzymes are often absent or low in activity (Schwartz R E, et al., Biotechnol Adv 2014, pii: S0734-9750(14)00005-6); 6. the above defects can be greatly improved by the introduction of exogenous genes, however, the introduced exogenous genes may change the gene structure, which leads to increased risk of carcinogenesis, making it impractical for clinical application; and 7. the proliferation, passage, cryopreservation, resuscitation, etc. of the differentiated hepatocytes are all closely related to clinical applications, but almost all reports have not mentioned these issues. Therefore, hepatocytes obtained by differentiating ES/iPS cells using the existing differentiation methods cannot be practically applied to clinical hepatocyte transplantation and bioartificial liver replacement therapy, because they cannot meet the clinical requirements. In 2013, Takebe, a Japanese scientist, obtained “liver bud”-liver microtissues by co-culturing iPSCs and MSCs with vascular endothelial cells (Takebe T, et al., Nature 2013, 499(7459): 481-4). The microtissues significantly improved both the metabolism and the survival rate of mice with liver injury. However, it was not reported that the “liver bud” tissues of this kind have the urea synthesis function of normal liver tissues or hepatocytes; and whether the method can be applied to the differentiation of human hepatocytes is still unknown, and there is a certain distance from the actual application. However, this finding provides a new idea for the clinical application research and development of the differentiation of ES/iPS cells into hepatocytes.

In summary, in the field of directed induction of human stem cells to differentiate into hepatocytes, in-depth researches are still needed to obtain truly applicable methods and products capable of providing qualified human hepatocytes for medical and clinical use.

Chemically induced direct cell reprogramming is a process that alters cell fate by inducing and regulating cell signaling pathways, epigenetic and chemical biological changes with chemical factors, without altering the gene sequence of the cells. By using the direct cell reprogramming mechanism for reference, the present invention provides a method for directly differentiating human stem cells into hepatocytes with high purity and high quality through inducing and regulating with only chemical small molecules without stepwise induction and the use of cell growth factors, which method hasn't been reported in any literature.

### SUMMARY OF THE INVENTION

The present invention aims at providing a novel method for inducing direct differentiation of human stem cells, preferably human embryonic stem cells or induced pluripotent stem cells, into hepatocytes using only small molecules only through conventional culturing methods without stepwise induction and the use of various cell growth factors and a dedicated direct differentiation medium used in the method.

The mechanism that has been basically defined by the present invention is mainly multi-target induction of changes in GSK3β and TGFβ signaling pathways through combinations of two types of small molecules, i.e., GSK3β inhibitors and TGFβ inhibitors; and the synergistic effect of retinoids, thereby regulating the changes of stem cell signaling pathways and epigenetics, thus allowing direct differentiation of stem cells into hepatocytes. It should be noted that GSK3β inhibitors and TGFβ inhibitors comprise a series of small molecules, which can be divided into two types but have the same function or induce the same target, and different combinations formed thereby can all induce stem cells to differentiate directly into hepatocytes to varying degrees. Therefore, the present invention claims small molecule compounds of the same type that have the same function or induce the same target, thus having the same effect on the same signaling pathway, as well as combinations of small molecules capable of inducing and regulating stem cells to differentiate directly into hepatocytes consisting of the small molecule compounds.

In a first aspect of the present invention, there is provided a medium for inducing directed differentiation of human stem cells into hepatocytes directly, the medium comprising: a cell differentiation minimal medium; and

a GSK3β inhibitor with a final concentration of 0.5-8 uM;

a TGFβ inhibitor with a final concentration of 0.1-10 uM; and

a retinoid with a final concentration of 0.001-10 uM;

wherein the medium can induce directed differentiation of human stem cells into hepatocytes directly, thereby obtaining human liver precursor cells or liver mature cells.

In one preferred embodiment, in the medium:

the GSK3β inhibitor is present at a final concentration of 0.5-5 uM;

the TGFβ inhibitor is present at a final concentration of 0.5-8 uM; and

the retinoid is present at a final concentration of 0.01-5 uM.

In another preferred embodiment, in the medium:

the GSK3β inhibitor is CHIR-99021 or/and CHIR-98014 with a final concentration of 0.5-8 uM, preferably 0.5-5 uM;

the TGFβ inhibitor is SB431542 or/and A83-01 or/and RepSox with a final concentration of 0.1-10 uM, preferably 0.5-8 uM; and

the retinoid is retinoic acid with a final concentration of 0.001-10 uM, preferably 0.01-5 uM.

In another preferred embodiment, one or more components can be also further to the medium, the components are selected from the group consisting of:
a Rock inhibitor: which is present at a final concentration of 0.5-50 uM, preferably 1-20 uM; and/or
hepatocyte which is present at a final concentration of 5-100
growth factor ng/ml, preferably 5-40 ng/ml; and/or
(HGF):
oncostatin M: which is present at a final concentration of 1-100 ng/ml, preferably 5-50 ng/ml; and/or
dexamethasone: which is present at a final concentration of 0.5-20 uM, preferably 2.5-10 uM;

wherein the medium to which the above components are added can increase the cell survival rate or promote the differentiation and maturation of ES/iPS cells into hepatocytes and maintain the growth of liver mature cells.

In another preferred embodiment, in the medium, the GSK3β inhibitor includes GSK3β signaling pathway inhibitors or compounds of the same type that have the same function or induce the same target and are represented by CHIR-99021, BIO, AZD2858, TWS119, CHIR-98014, etc., or combinations thereof, preferably the GSK3β inhibitor is CHIR-99021;

the TGFβ inhibitor includes TGFβ signaling pathway inhibitors or compounds of the same type that have the same function or induce the same target and are represented by SB431542, A83-01, SB525334, LY2109761, RepSox, etc., or combinations thereof, preferably the TGFβ inhibitor is SB431542 or/and A83-01;

the retinoid is natural or synthetic and includes retinoid differentiation agents or compounds of the same type that have the same function or induce the same target and are represented by retinoic acid (RA, also known as all trans retinoic acid (ATRA)), 13-cis retinoic acid (13-CRA), 9-cis-retinoic acid (9-CRA), etc., or combinations thereof, preferably the retinoid is retinoic acid (RA); and

the Rock inhibitor includes Rock signaling pathway inhibitors or compounds of the same type that have the same function or induce the same target and are represented by Y-27632 (alternative name: Y-27632 2HCl), GSK429286A, RKI-1447, etc., or combinations thereof, preferably the Rock inhibitor is Y-27632.

In another preferred embodiment, in the medium, the cell differentiation minimal medium is a minimal cell medium to which 0.5% N2, 1% B27, 1% Non-AA and 1% Sodium pyruvate, preferably 1% penicillin are added; wherein, the content in percentage of each component in the cell differentiation minimal medium can also be increased or decreased by 50%, preferably by 30%, more preferably by 20%, such as 10% and 5%; preferably, the minimal cell medium includes, but is not limited to: DMEM/F12, MEM, DMEM, RPMI1640, Neuronal basal, Fischers or the like.

In another aspect of the present invention, there is provided a composition comprising a GSK3β inhibitor, a TGFβ inhibitor and a retinoid and use of the medium for inducing directed differentiation of human stem cells into hepatocytes directly, thereby obtaining human liver precursor cells or liver mature cells; preferably, the composition also comprises a component selected from the group of a Rock inhibitor, hepatocyte growth factor, oncostatin M or dexamethasone.

In one preferred embodiment, in the composition, the GSK3β inhibitor, the TGFβ inhibitor and the retinoid are present at a molar ratio (which can be also converted to weight ratio) of (0.5-8): (0.1-10): (0.001-10), preferably (0.5-5): (0.5-8): (0.01-5).

In another aspect of the present invention, there is provided a kit for inducing directed differentiation of human stem cells into hepatocytes directly, comprising a GSK3β inhibitor, a TGFβ inhibitor and a retinoid, for use in inducing directed differentiation of human stem cells into hepatocytes directly; preferably, the kit further comprises a component selected from the group of a Rock inhibitor, hepatocyte growth factor, oncostatin M or dexamethasone, for use in increasing the cell survival rate or promoting the differentiation of ES/iPS cells into functional liver mature cells and maintaining the growth of liver mature cells during inducing directed differentiation of human stem cells into hepatocytes directly; or, the kit comprises any of the aforementioned media.

In another aspect of the present invention, there is provided a method for inducing directed differentiation of human stem cells into hepatocytes directly, the method comprising: using any of the aforementioned media to induce directed differentiation of human stem cells into hepatocytes directly; preferably, the method comprises the following steps:

(1) initiation of human liver precursor cell differentiation: coating a culture plate with one of matrigel, rat tail gel, gelatin, fibronectin and vitronectin for 30 minutes to 24 hours; then suspending human stem cells in the hepatocyte differentiation medium or a hepatocyte differentiation-strengthening medium, preferably in the hepatocyte differentiation medium and plating the suspension in the culture plate; and culturing the culture plate at 37° C.±1° C. and 5% CO₂, with the medium changed once every 72 hours;

(2) subculturing: subculturing the cells at a ratio of 1:2-1:5 if the confluence reaches 90%;

subculturing steps: digesting the differentiated human ES/iPS cells into single cells with a digestion solution comprising trypsin, EDTA, Acutase, Tryple E, etc., and resuspending and subculturing the single cells at a ratio of 1:2-1:5; and subculturing the differentiated cells according to the method described in step (1);

(3) differentiation to obtain human liver precursor cells: differentiation culture of the cells for 10-15 days according to the method described in steps (1) and (2) to obtain human liver precursor cells which can be used for cryopreservation, resuscitation and passage and can be further induced to differentiate into functional human liver mature cells; preferably, the subculturing method comprises: subculturing the differentiated human liver precursor cells according to the method described in step (2); and

(4) maturation culture of human liver precursor cells: further differentiation culture of the human liver precursor cells obtained by the differentiation culture in step (3) in the hepatocyte differentiation medium or a hepatocyte differentiation-strengthening medium, preferably in the hepatocyte differentiation-strengthening medium, for 7-15 days at 37° C.±1° C. and 5% CO₂ to obtain functional human liver mature cells which can be used for cryopreservation, resuscitation and limited passage.

In another preferred embodiment, the human stem cells include, but are not limited to: human embryonic stem cells, induced pluripotent stem cells, mesenchymal stem cells, adipose stem cells, umbilical cord blood stem cells and other human stem cells having multi-lineage differentiation potential; preferably, the human stem cells are human embryonic stem cells or induced pluripotent stem cells.

Other aspects of the present invention will be apparent to those skilled in the art from the disclosure herein.

### BRIEF DESCRIPTION OF THE DRAWINGS

FIG. 1 shows comparison of morphology of human ES cells with differentiated liver precursor cells; left panel: human ES cells; right panel: liver precursor cells differentiated from human ES cells.

FIG. 2 shows high density and low density subculture of hepatocytes differentiated from human ES cells.

FIG. 3 shows comparison of morphology of human ES cells with differentiated liver mature cells and human primary hepatocytes; left panel: human ES cells; middle panel: liver mature cells differentiated from human ES cells; right panel: human primary hepatocytes.

FIG. 4 shows results of flow cytometric analysis of hepatocyte-specific marker staining of liver mature cells differentiated from human ES cells. The results show that there is a very high proportion of differentiated liver mature cells that are positive for the hepatocyte-specific marker.

FIG. 5 shows comparison of albumin production of liver mature cells differentiated from human iPS cells with that of primary hepatocytes, in which PHH represents primary hepatocytes; iPSC-Hep represents liver mature cells differentiated from human iPS cells; and C1-C4 represent 4 culturing conditions.

FIG. 6 shows comparison of urea production of liver mature cells differentiated from human iPS cells with that of primary hepatocytes, in which PHH represents primary hepatocytes; iPSC-Hep represents liver mature cells differentiated from human iPS cells; and C1-C4 represent 4 culturing conditions. Urea is derived from blood ammonia in the blood, and its production reflects the detoxification ability of hepatocytes.

FIG. 7 shows glycogen staining of liver mature cells differentiated from human ES cells. The degree of staining reflects the ability of hepatocytes to store glycogen.

FIG. 8 shows activity induction of P450 enzymes (CYP3A4 and CYP1A2) of liver mature cells differentiated from human ES cells. Left panel: the activity of CYP3A4 is increased when induced by different concentrations of rifampicin; right panel: the activity of CYP1A2 is increased when induced by different concentrations of omeprazole.

FIG. 9 shows comparison of morphology of human ES cells with directly differentiated liver precursor cells and hepatocytes; left panel: human ES cells; middle panel: liver precursor cells differentiating for 6 days; right panel: hepatocytes differentiating for 15 days. FIG. 9 shows that the combination of small molecules comprising GSK3β inhibitor BIO, a TGFβ inhibitor and retinoic acid can also induce direct differentiation of stem cells into liver precursor cells and liver mature cells.

FIG. 10 shows comparison of morphology of human iPS cells with directly differentiated liver precursor cells and hepatocytes; left panel: human iPS cells; middle panel: liver precursor cells differentiating for 6 days; right panel: hepatocytes differentiating for 20 days. FIG. 10 shows that the combination of small molecules comprising TGFβ inhibitor RepSox, a GSK3β inhibitor and retinoic acid can also induce direct differentiation of stem cells into liver precursor cells and liver mature cells.

FIG. 11 shows comparison of morphology of human ES cells with directly differentiated liver precursor cells and hepatocytes; left panel: human ES cells; middle panel: liver precursor cells differentiating for 6 days; right panel: hepatocytes differentiating for 16 days. FIG. 11 shows that the combination of small molecules comprising GSK3β inhibitor CHIR-98014, TGFβ inhibitor SB525334 and retinoic acid can also induce direct differentiation of stem cells into liver precursor cells and liver mature cells.

### DETAILED DESCRIPTION OF EMBODIMENTS

The present inventors have conducted in-depth researches and developed a novel method for inducing directed differentiation of human stem cells, preferably human embryonic stem cells (ES cells) or induced pluripotent stem cells (iPS cells), into hepatocytes directly and a dedicated direct differentiation medium used in the method. In the method, no exogenous genes are needed to be introduced into stem cells, no growth factors are needed, no stepwise induction is needed, and differentiation of stem cells into hepatocytes can be achieved by conventional culturing. The differentiated human hepatocytes obtained have the typical characteristics of human hepatocytes; the obtained human liver precursor cells can be maintained for a long period of time, can be used for cryopreservation, resuscitation, proliferation and passage, and can be then subjected to maturation, differentiation and culture to obtain functional human liver mature cells; and the obtained functional human liver mature cells can be used for cryopreservation, resuscitation, limited passage and maintenance culture. Moreover, the method has simple culturing conditions, low cost, safety and stability.

As used in the present invention, the term “contain (contains, contained and containing)” or “include (includes, included and including)” includes “comprise (comprises, comprised and comprising)”, “consist (consists, consisted and consisting) mainly of”, “consist (consists, consisted and consisting) essentially of” and “consist (consists, consisted and consisting) of”.

In the present invention, human stem cells, which are subjected to directed induction for direct differentiation include but not limited to: human embryonic stem cells, induced pluripotent stem cells, mesenchymal stem cells, adipose stem cells, umbilical cord blood stem cells and other human stem cells having multi-lineage differentiation potential; preferably, the human stem cells are human embryonic stem cells or induced pluripotent stem cells.

### Media

The present invention provides a novel method for inducing directed differentiation of human stem cells, preferably human embryonic stem cells or induced pluripotent stem cells, into hepatocytes directly and a dedicated direct differentiation medium used in the method. The dedicated direct differentiation medium includes “hepatocyte differentiation medium” and “hepatocyte differentiation-strengthening medium”.

The hepatocyte differentiation medium comprises a GSK3β inhibitor, a TGFβ inhibitor and a retinoid. The above components are added to the cell differentiation minimal medium at a suitable ratio, and directed differentiation of human stem cells into hepatocytes directly can be induced.

As used in the present invention, the “GSK3β inhibitor” refers to a generic term for inhibitors capable of inhibiting the GSK3β signaling pathways in cells, including but not limited to: inhibitors of the same type that have the same function or induce the same target and are represented by CHIR-99021, BIO, AZD2858, TWS119, CHIR-98014, etc.;

wherein CHIR-99021 (CT99021) is an inhibitor for GSK-3α and GSK-3β with an IC50 of 10 nM and 6.7 nM, respectively, and 500 times more inhibitory on GSK-3α and GSK-3β than on CDC2, ERK2 and other kinases;

CHIR-99021 (CT99021) HCl, which is the hydrochloride salt of CHIR-99021, is an inhibitor for GSK-3α/β with an IC50 of 10 nM/6.7 nM in cell-free assays, and can be used to distinguish GSK-3 and its closest homologues Cdc2 and ERK2;

BIO is a specific GSK-3 inhibitor, and has an IC50 of 5 nM for GSK-3α/β in cell-free assays;

AZD2858 is a selective GSK-3 inhibitor with an IC50 of 68 nM, and enhances the Wnt signaling pathway;

TWS119 is a GSK-3β inhibitor, and has an IC50 of 30 nM in cell-free assays;

CHIR-98014 is a potent inhibitor for GSK-3α/β, and has an IC50 of 0.65 nM/0.58 nM in cell-free assays;

Tideglusib is an irreversible, non-ATP-competitive GSK-3β inhibitor, and has an IC50 of 60 nM in cell-free assays;

AR-A014418 is an ATP-competitive and selective GSK3β inhibitor, and has an IC50 of 104 nM and a Ki of 38 nM in cell-free assays;

LY2090314 is a potent GSK-3 inhibitor for GSK-3α/β with an IC50 of 1.5 nM/0.9 nM;

SB216763 is a potent, selective inhibitor for GSK-3α/β with an IC50 of 34.3 nM; and

AZD1080 is an orally bioavailable and selective GSK3 inhibitor that is permeable to the brain, inhibits human GSK3a and GSK3β with a Ki of 6.9 nM and 31 nM, respectively, and is 14 times or above more selective on GSK3 than on CDK2, CDK5, CDK1 and Erk2.

As a preferred embodiment of the present invention, the GSK3β inhibitor is CHIR-99021 (alternative name: CT99021), which has a molecular structure as shown in the following formula (I):

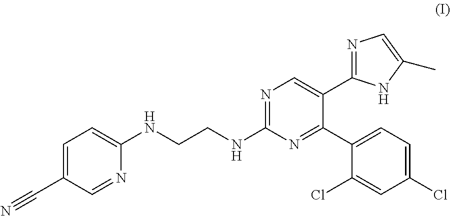 [SMILES: Cc1cnc(-c2cnc(NCCNc3ccc(C#N)cn3)nc2-c2ccc(Cl)cc2Cl)[nH]1]

As used in the present invention, the “TGFβ inhibitor” refers to a generic term for inhibitors capable of inhibiting the TGFβ signaling pathways in cells, including but not limited to: SB431542, A83-01, SB525334, LY2109761, RepSox and other inhibitors of the same type that have the same function;

wherein SB-431542 is a potent and selective ALK5 inhibitor with an IC50 of 94 nM, and is 100 times more inhibitory on ALK5 than on p38, MAPK and other kinases;

A 83-01 is an inhibitor for ALK5, ALK4 and ALK7 with an IC50 of 12, 45, and 7.5 nM, respectively;

SB525334 is a potent and selective TGFβ receptor I (ALK5) inhibitor with an IC50 of 14.3 nM in cell-free assays, and has a effect on ALK4 4 times lower than on ALK5, and has no activity on ALK2, ALK3 and ALK6;

LY2109761 is a novel and selective TGF-β receptor type I/II (TβRI/II) dual inhibitor with a Ki of 38 nM and 300 nM, respectively, in cell-free assays;

RepSox is a potent and selective TGFβR-1/ALK5 inhibitor, and influences the binding of ATP to ALK5 and ALK5 autophosphorylation with an IC50 of 23 nM and 4 nM, respectively, in cell-free assays;

SD-208 is a selective TGF-βRI (ALK5) inhibitor with an IC50 of 48 nM, and is 100 times or above more selective on TGF-βRI than on TGF-βRII;

GW788388 is a potent and selective ALK5 inhibitor with an IC50 of 18 nM in cell-free assays, also inhibits the activity of TGF-β type II receptors and activin type II receptors, but does not inhibit the activity of BMP type II receptors;

SB505124 is a selective TGFβR inhibitor for ALK4 and ALK5 with an IC50 of 129 nM and 47 nM, respectively, in cell-free assays, also inhibits ALK7, but does not inhibit ALK1, ALK2, ALK3 or ALK6; and

EW-7197 is a highly potent, selective and orally bioavailable TGF-beta receptor ALK4/ALK5 inhibitor with an IC50 of 13 nM and 11 nM, respectively.

As a preferred embodiment of the present invention, the TGFβ inhibitor is SB 431542 (or referred to as SB-431542), which has a molecular structure as shown in the following formula (II):

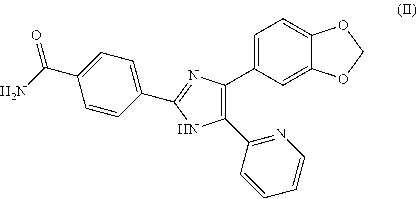 [SMILES: NC(=O)c1ccc(-c2nc(-c3ccc4c(c3)OCO4)c(-c3ccccn3)[nH]2)cc1]

As a preferred embodiment of the present invention, the TGFβ inhibitor is A83-01 (or referred to as A8301), which has a molecular structure as shown in the following formula (III):

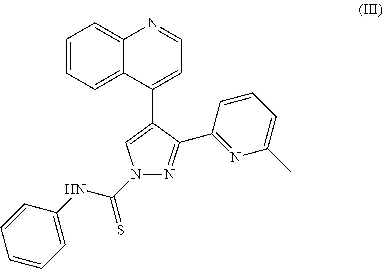 [SMILES: Cc1cccc(-c2nn(C(=S)Nc3ccccc3)cc2-c2ccnc3ccccc23)n1]

As used in the present invention, the retinoid includes retinoid differentiation agents or compounds of the same type that have the same function or induce the same target and are represented by retinoic acid (RA) (alternative name: all trans retinoic acid (ATRA)), 13-cis retinoic acid (13-CRA), 9-cis-retinoic acid (9-CRA), etc., or combinations thereof;

and retinoids, including both natural retinoids and synthetic retinoids, are a group of oxidative metabolites or derivatives of vitamin A (retinol) and synthetics with similar structures to vitamin A. Retinoids mainly include retinoic acid (RA) (alternative name: tretinoin and all trans retinoic acid (ATRA)), 13-cis retinoic acid (13-CRA), 9-cis-retinoic acid (9-CRA), isotretinoin, fenretinide, acitretin, etretinate, tazarotene, adapalene, TTNPB, CD437, targretin, etc. Retinoids are characterized by regulating differentiation, proliferation and apoptosis of many types of cells in vitro and in vivo. Many of the retinoids and their isomer derivatives have the same or similar functions. Therefore, they have become a class of drugs which are the most important differentiation-inducing agents and have been used for the clinical treatment of skin diseases.

As a preferred embodiment of the present invention, the retinoic acid (RA), or referred to as all trans retinoic acid (ATRA), tretinoin, vitamin A acid, vitamin formic acid, retinoc acid, all trans tretinoin, vitamin A formic acid, has a molecular structure as shown in the following formula (IV):

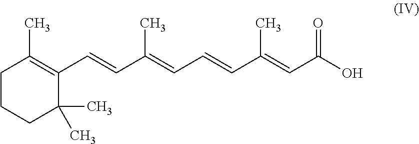 [SMILES: CC1=C(/C=C/C(C)=C/C=C/C(C)=C/C(=O)O)C(C)(C)CCC1]

As used in the present invention, the “Rock inhibitor” refers to a generic term for inhibitors capable of inhibiting the Rock signaling pathways in cells, including but not limited to: inhibitors of the same type that have the same function or induce the same target and are represented by Y-27632, GSK429286A, RKI-1447, etc.;

wherein Y-27632 (Y-27632 2HCl) is a selective ROCK1 (p160ROCK) inhibitor with a of 140 nM in cell-free assays, and is 200 times or above more inhibitory on ROCK1 than on other kinases including PKC, cAMP-dependent protein kinases, MLCK and PAK;

GSK429286A is a selective inhibitor for ROCK1 and ROCK2 with an IC50 of 14 nM and 63 nM, respectively;

RKI-1447 is a potent inhibitor for ROCK1 and ROCK2 with an IC50 of 14.5 nM and 6.2 nM, respectively, and has anti-invasion and anti-tumor activity;

Thiazovivin is a novel ROCK inhibitor with an IC50 of 0.5 μM in cell-free assays, and promotes the survival of human embryonic stem cells (hESCs) after single cell isolation; and

RO-3306 is an ATP-competitive and selective CDK1 inhibitor with a of 20 nM, and is 15 times or above more selective on CDK1 than on various other human kinases.

As a preferred embodiment of the present invention, the Rock inhibitor is Y-27632 (Y-27632 2HCl) (alternative name: Y-27632 dihydrochloride or Y-27632 2HCl), which has a molecular structure as shown in the following formula (V).

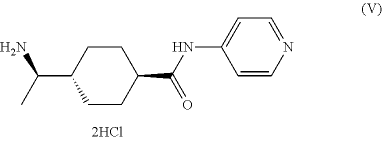 [SMILES: C[C@@H](N)[C@H]1CC[C@H](C(=O)Nc2ccncc2)CC1]

In the previous studies, the present inventors proposed that down-regulation of the GSK3β signaling pathway and the TGFβ signaling pathway in combination with retinoic acid (RA) can promote directed differentiation of human pluripotent stem cells into hepatocytes directly in the art for the first time. It should be understood that, in addition to the specific GSK3β inhibitors and TGFβ inhibitors listed in the examples of the present invention, other inhibitors that can inhibit the GSK3β signaling pathway and the TGFβ signaling pathway can also achieve the same technical effects, which should also be included in the present invention.

Likewise, in addition to the specific differentiation-inducing agent retinoic acid (RA) and Rock inhibitors listed in the examples of the present invention, other retinoids and inhibitors for the Rock signaling pathway that have the same function or induce the same target can also achieve the same technical effects, which should also be included in the present invention.

Equivalent compounds, analogues, derivatives, and/or salts, hydrates or precursors of the various compounds mentioned above that have the same function or induce the same target are also included in the present invention. Biochemical reagents and pharmaceutical preparations prepared from the various compounds mentioned above are also included in the present invention.

Analogues of the compounds include, but are not limited to: isomers and racemates of the compounds. The compounds have one or more asymmetric centers. Therefore, these compounds may exist as racemic mixtures, individual enantiomers, individual diastereomers, diastereomeric mixtures, cis- or trans-isomers.

The “salts” include, but are not limited to: (1) salts formed with such inorganic acids as hydrochloric acid, sulfuric acid, nitric acid, phosphoric acid, etc.; and (2) salts formed with such organic acids as acetic acid, oxalic acid, succinic acid, tartaric acid, methanesulfonic acid, maleic acid, arginine or the like. Other salts include salts formed with alkali or alkaline earth metals such as sodium, potassium, calcium or magnesium, etc.

The “precursor of a compound” refers to a precursor that can be converted into any of the above compounds in a medium after the precursor is applied or treated in a suitable manner, or to a salt or solution formed with any of the above compounds.

As used in the present invention, the “cell differentiation minimal medium” is a minimal nutrient maintenance medium used in the art for differentiation culture of human stem cells, such as one that is commonly used for differentiation culture of human embryonic stem cells or induced pluripotent stem cells. Without the addition of the hepatocyte differentiation medium and the hepatocyte differentiation-strengthening medium according to the present invention or the active components of the composition therein, using the “cell differentiation minimal medium” cannot allow directed differentiation of human embryonic stem cells or induced pluripotent stem cells into hepatocytes directly.

As a preferred embodiment of the present invention, the “cell differentiation minimal medium” is a minimal cell medium to which 0.5% N2, 1% B27, 1% Non-AA and 1% Sodium pyruvate, preferably also 1% penicillin (percentages are in v/v are added, wherein each content in percentage can also be increased or decreased by 50%, preferably by 30%, more preferably by 20%, such as 10% and 5%). Alternatively, the “cell differentiation minimal medium” are also be commercially available. The minimal cell medium may be, without limitation: DMEM/F12, MEM, DMEM, RPMI1640, Neuronal basal, Fischers or the like. It should be understood that those skilled in the art are familiar with the formulation or purchase channels of the minimal cell medium, and therefore the minimal cell medium is not limited to those exemplified in the present invention.

As a preferred embodiment of the present invention, the “hepatocyte differentiation medium” according to the present invention is formulated as follows:

adding the following components to a cell differentiation minimal medium:

(1) GSK3β inhibitor CHIR-99021 with a final concentration of 0.5-8 uM, preferably 0.5-5 uM;

(2) TGFβ inhibitor SB431542 or/and A83-01 with a final concentration of 0.1-10 uM, preferably 0.5-8 uM; and

(3) retinoic acid (RA) with a final concentration of 0.001-10 uM, preferably 0.01-5 uM.

According to the formula above, the dedicated “hepatocyte differentiation medium” of the present invention can be obtained that is used in the “novel method for inducing directed differentiation of human stem cells into hepatocytes”.

On the basis of the formula of the “hepatocyte differentiation medium” above, any one or more of the following components can be added to obtain the “hepatocyte differentiation-strengthening medium”:

(1) a Rock inhibitor (which is described in detail above) or preferably Rock inhibitor Y-27632 with a final concentration of 0.5-50 uM, preferably 1-20 uM; and/or

(2) hepatocyte growth factor (HGF) with a final concentration of 5-100 ng/ml, preferably 5-40 ng/ml; and/or

(3) oncostatin M with a final concentration of 1-100 ng/ml, preferably 5-50 ng/ml; and/or

(4) dexamethasone with a final concentration of 0.5-20 M, preferably 2.5-10 M.

According to the formula above, the “hepatocyte differentiation-strengthening medium” can be obtained, which can increase the cell survival rate during inducing direct differentiation of human stem cells into hepatocytes or promote ES/iPS cells or liver precursor cells to continue to differentiate into functional liver mature cells or enhance certain biological behaviors and functions of differentiated hepatocytes, and can maintain the growth of liver mature cells for a long period of time.

### Culturing Methods

The present invention also discloses a novel method for inducing direct differentiation of human stem cells, preferably human embryonic stem cells or induced pluripotent stem cells, into hepatocytes using only combinations of small molecules. The method involves inducing differentiation and culturing of human ES/iPS cells in the “hepatocyte differentiation medium” and/or “hepatocyte differentiation-strengthening medium” of the present invention to obtain liver precursor cells or liver mature cells with high purity.

As a preferred embodiment of the present invention, the method for culturing liver precursor cells comprises: coating a culture plate for 1-24 hours; then adding and suspending human ES/iPS cells in the hepatocyte differentiation medium or hepatocyte differentiation-strengthening medium, preferably the hepatocyte differentiation medium and plating the suspension in the culture plate; and culturing the culture plate at 37° C.±1° C. and 5% CO2 for 10-15 days, with the medium changed every 72±1 hours, to obtain liver precursor cells.

The method for coating a culture plate is well known in the art, and materials that can be used to coat a culture plate include, but are not limited to: matrigel, rat tail gel, gelatin, fibronectin, vitronectin, etc., one of which can be selected for coating.

During differentiation culture of human ES/iPS cells, if the confluence reaches 85%-90%, the cells can be subcultured at a ratio of 1:2-1:5. As a preferred embodiment of the present invention, the subculturing step comprises: digesting human ES/iPS cells during differentiation culture into single cells with a digestion solution, and resuspending and subculturing the differentiated cells at a ratio of 1:2-1:5. The digestion solution may be a solution comprising one or more digestive enzymes like trypsin, EDTA, Acutase, Tryple E, etc.

The obtained liver precursor cells can be used for cryopreservation, resuscitation and passage, and can be further induced to be functional human liver mature cells.

As a preferred embodiment of the present invention, the method for inducing liver precursor cells to be functional human liver mature cells comprises: adding and suspending liver precursor cells in the hepatocyte differentiation medium or hepatocyte differentiation-strengthening medium, preferably the hepatocyte differentiation-strengthening medium; and then subjecting the cells to differentiation and maturation culture at 37° C.±1° C. and 5% CO2 for 7-15 days to obtain functional liver mature cells.

The obtained liver mature cells by the method of the present invention can be used for cryopreservation, resuscitation, limited passage and maintenance culture for a long period of time. They can also be applied to cell transplantation therapy for liver diseases, bioartificial liver construction, liver toxicity testing, efficacy assessment and target identification of new drugs. They can provide adequate sources of hepatocytes or hepatocyte models for basic researches and clinical applications of biology, medicine and pharmacy. Their induced differentiation process can also provide the best platform for researching the development and differentiation processes of human liver cells. They have very broad application prospects.

In addition, it should also be understood that the human embryonic stem cells involved in the present invention are all commercially available embryonic stem cell lines, such as those listed in Table 1, and do not involve the use of human embryos.

TABLE 1
Commercialized human embryonic stem cell lines

| Provider | Submit number | NTR number |
|---|---|---|
| BresaGen, Inc. | hESBGN-01, | BG01, |
| | hESBGN-02, | BG02, |
| | hESBGN-03, | BG03, |
| | hESBGN-04 | BG04 |
| Cellartis AB | Sahlgrenska 1, | SA01, |
| | Sahlgrenska 2, | SA02, |
| | Sahlgrenska 3 | SA03 |
| ES Cell | HES-1, | ES01, |
| International | HES-2, | ES02, |
| | HES-3, | ES03, |
| | HES-4, | ES04, |
| | HES-5, | ES05, |
| | HES-6 | ES06 |
| Technion-Israel | I 3, | TE03, |
| Institute of | I 3.2, | TE32, |
| Technology | I 3.3, | TE33, |
| | I 4, | TE04, |
| | I 6, | TE06, |
| | I 6.2, | TE62, |
| | I 3, | TE07, |
| | I 3.2 | TE72 |
| University of | HSF-1, | UC01, |
| California, San | HSF-6 | UC06 |
| Francisco | | |
| Wisconsin Alumni | H1, | WA01, |
| Research | H7, | WA07, |
| Foundation | H9, | WA09, |
| (WiCell Research | H13, | WA13, |
| Institute) | H14 | WA14 |

As early as 1998, human embryonic stem cell lines and embryonic germ cell lines had been successfully established. For example, in 1998, the team led by Thomson finally established 5 human ES cell lines from 14 blastulas: H1, H13, H14, H7 and H19; and the team led by Gearhart isolated primary stem cells from the gonadal ridges and mesenteries of aborted fetuses aged 5-9 weeks in hopes of avoiding the ethical inconvenience caused by the direct use of embryos. See CHAO Lan, “Research Progress of Human Embryonic Stem Cells”, Progress in Obstetrics and Gynecology, Vol. 12, No. 4, July 2003. Based on the above work, in February 2000, Wisconsin Alumni Research Foundation (WARF) established WiCell, an unofficial, non-profit affiliate that dispenses human embryonic stem cells to qualified scientists at low cost. In addition, institutions providing such ready-made human embryonic stem cells include NSCB (National Stem Cell Bank), ES CELL INTERNATIONAL, nov cell, TECHNION-HOME TO ISRAEL'S NOBEL SCIENTISTS, UCSF (University of California San Fransisco) and other institutions. Therefore, human embryonic stem cells can be obtained completely by other means “taken from human embryos”.

The beneficial technical effects of the method of the present invention lie in:

1. Only combinations of small molecules are used to multi-target-directed induce direct differentiation of human ES cells or iPS cells into hepatocytes only through conventional culturing without stepwise induction and using various expensive cell growth factors; and since small molecules are stable in nature, differentiation results are stable and safe, and the cost is greatly reduced.

2. Only combinations of small molecules are used to directed induce direct differentiation of human ES cells or iPS cells into hepatocytes without introducing exogenous genes and altering gene structures, thus avoiding experimental interference and carcinogenic risk caused by the introduction of exogenous genes and changes in gene structure (different from liver tumor cells, immortalized hepatocytes, and hepatocytes transformed by introducing exogenous genes into iPS cells).

3. The direct differentiation efficiency is high, that is to say, one million (1×10⁶) human ES or iPS cells can be converted to 5-10×10⁶ or more functional hepatocytes (which is not mentioned in any reports); and the differentiated hepatocytes are highly consistent with human primary hepatocytes in morphology and function.

4. The direct differentiation purity is high without the need for additional purification means, and the results of flow cytometric analysis (FACS) show that the purity of the differentiated hepatocytes obtained is as high as 90% or above; there are >90% (20%-60% in other reports generally) of hepatocytes obtained after differentiation culture that are positive for the surface marker Asgp specific for mature hepatocytes; there are 80% or more of cells that are positive for various other hepatocyte-specific immune markers, such as ALB, CYP3A and HNF4a, showing that the differentiated hepatocytes have high quality; and the cost is effectively reduced.

5. The directly differentiated hepatocytes are well universal and reproducible, and the present inventors have verified that 9 human ES/iPS cell lines (including 2 human ES cell lines and 7 human iPS cell lines) are all differentiated into hepatocytes with consistent morphology and function.

6. The directly differentiated hepatocytes have many functions unique to hepatocytes, such as albumin production, urea synthesis, activity induction of P450 enzymes (CYP enzymes: CYP3A4 and CYP1A2), glycogen storage, etc. Thus, the hepatocytes obtained by the method of the present invention are functional hepatocytes.

7. The method of the present invention is a culturing method free of animal-derived components, doesn't use animal feeder cells to culture ES/iPS cells, and can be directly applied to mass production and clinical application according to GMP standard, which features have not been reported; and the method does not have the problem of contamination with animal-derived materials.

8. The direct differentiation technology does not require the formation of embryo bodies (EBs), so the method is simple, easy to operate, low in cost and suitable for mass production.

9. The differentiation stage is clear and the quality is easy to control.

10. The liver precursor cells obtained by direct differentiation can be used for proliferation, passage, cryopreservation and resuscitation, and can be then induced to differentiate into mature functional hepatocytes; and the obtained liver mature cells can be used for cryopreservation, resuscitation, limited passage and maintenance culture.

The present invention is further illustrated with reference to the specific examples below. It should be understood that these examples are only for illustrating the present invention and are not intended to limit the scope of the present invention. The experimental methods that do not specify the specific conditions in the following examples are generally performed according to conventional conditions such as those described in J. Sambrook et al. (editors), Molecular Cloning: A Laboratory Manual, 3rd Edition, Science Press, 2002, or according to the manufacturer's recommended conditions

### Example 1. Formulation of Media for Inducing Direct Differentiation of Human Embryonic Stem Cells or Induced Pluripotent Stem Cells into Hepatocytes

### 1. Formulation of a Cell Differentiation Minimal Medium

The cell differentiation minimal medium is prepared according to conventional methods. That is, a mixed solution (100×) of 0.5% N2, 1% B27, 1% Non-AA, 1% Sodium pyruvate and 1% penicillin was added to DMEM/F12 (a minimal cell medium). The percentages are all in v/v.

### 2. Formulation of Hepatocyte Differentiation Media

### (1) Hepatocyte Differentiation Medium 1

To the above “cell differentiation minimal medium”, the following components with the indicated final concentrations were added:

GSK3β inhibitor CHIR-99021: 2 uM;

TGF inhibitor SB431542: 5 uM; and

retinoic acid (RA): 2 uM.

### (2) Hepatocyte Differentiation Medium 2

To the above “cell differentiation minimal medium”, the following components with the indicated final concentrations were added:

GSK3β inhibitor CHIR-99021: 2 uM;

TGF inhibitor A83-01: 3 uM; and

retinoic acid (RA): 5 uM.

### (3) Hepatocyte Differentiation Medium 3

To the above “cell differentiation minimal medium”, the following components with the indicated final concentrations were added:

GSK3β inhibitor CHIR-99021: 1 uM;

TGFβ inhibitor SB431542: 2 uM; and

retinoic acid (RA): 0.1 uM.

### (4) Hepatocyte Differentiation Medium 4

To the above “cell differentiation minimal medium”, the following components with the indicated final concentrations were added:

GSK3β inhibitor CHIR-99021: 2 uM;

TGFβ inhibitor A83-01: 3 uM; and

retinoic acid (RA): 0.5 uM.

### (5) Hepatocyte Differentiation Medium 5

GSK3β inhibitor CHIR-99021: 3 uM;

TGFβ inhibitor SB431542: 5 uM; and

retinoic acid (RA): 3 uM.

### (6) Hepatocyte Differentiation Medium 6

GSK3β inhibitor BIO: 3 uM;

TGFβ inhibitor SB431542: 5 uM; and

retinoic acid (RA): 2 uM.

### (7) Hepatocyte Differentiation Medium 7

GSK3β inhibitor CHIR-99021: 3 uM;

TGFβ inhibitor RepSox: 5 uM; and

retinoic acid (RA): 1 uM.

### (8) Hepatocyte Differentiation Medium 8

GSK3β inhibitor CHIR-98014: 2 uM;

TGFβ inhibitor SB525334: 6 uM; and

retinoic acid (RA): 5 uM.

### 3. Formulation of Hepatocyte Differentiation-Strengthening Media

### (1) Hepatocyte Differentiation-Strengthening Medium 1

On the basis of the formula of the above “hepatocyte differentiation medium 1”, the following components were added:

Rock inhibitor (Y-27632) with a final concentration of 10 uM; and

dexamethasone with a final concentration of 5 uM.

### (2) Hepatocyte Differentiation-Strengthening Medium 2

On the basis of the formula of the above “hepatocyte differentiation medium 3”, the following components were added:

Rock inhibitor (Y-27632) with a final concentration of 5 uM; and

oncostatin M with a final concentration of 20 ng/ml.

### (3) Hepatocyte Differentiation-Strengthening Medium 3

On the basis of the formula of the above “hepatocyte differentiation medium 4”, the following components were added:

Rock inhibitor (Y-27632) with a final concentration of 2.5 uM; and

human hepatocyte growth factor (HGF) with a final concentration of 20 ng/ml.

### (4) Hepatocyte Differentiation-Strengthening Medium 4

On the basis of the formula of the above “hepatocyte differentiation medium 2” or “hepatocyte differentiation medium or 8”, the following component was added:

Rock inhibitor (Y-27632) with a final concentration of 15 uM.

### Example 2. Differentiation Culture of Human Liver Precursor Cells and Culture of Liver Mature Cells Using Hepatocyte Differentiation Medium 1 and Hepatocyte Differentiation-Strengthening Medium 2

### 1. Initiation of Human Liver Precursor Cell Differentiation

A culture plate was coated with matrigel for 12 hours, hepatocyte differentiation medium 1 was added to the culture plate, human embryonic stem cells (ES) (see FIG. 1, left panel) were then suspended in “hepatocyte differentiation medium 1”, and the suspension was plated in the culture plate; and the culture plate was cultured at 37° C. and 5% CO2, with the medium changed every 72 hours.

Human liver precursor cells can be obtained after differentiation culture of human embryonic stem cells in hepatocyte differentiation medium 1 at 37° C. and 5% CO2 for 10-15 days. FIG. 1 shows comparison of morphology of human ES cells with liver precursor cells obtained from differentiation, in which right panel shows liver precursor cells obtained by differentiation culture for 15 days, which can be used for cryopreservation, resuscitation and passage, and can be further induced to be functional human liver mature cells.

### 2. Subculturing

During differentiation culture, if the confluence reached 90%, the cells during differentiation culture can be subcultured at a ratio of 1:2-1:5.

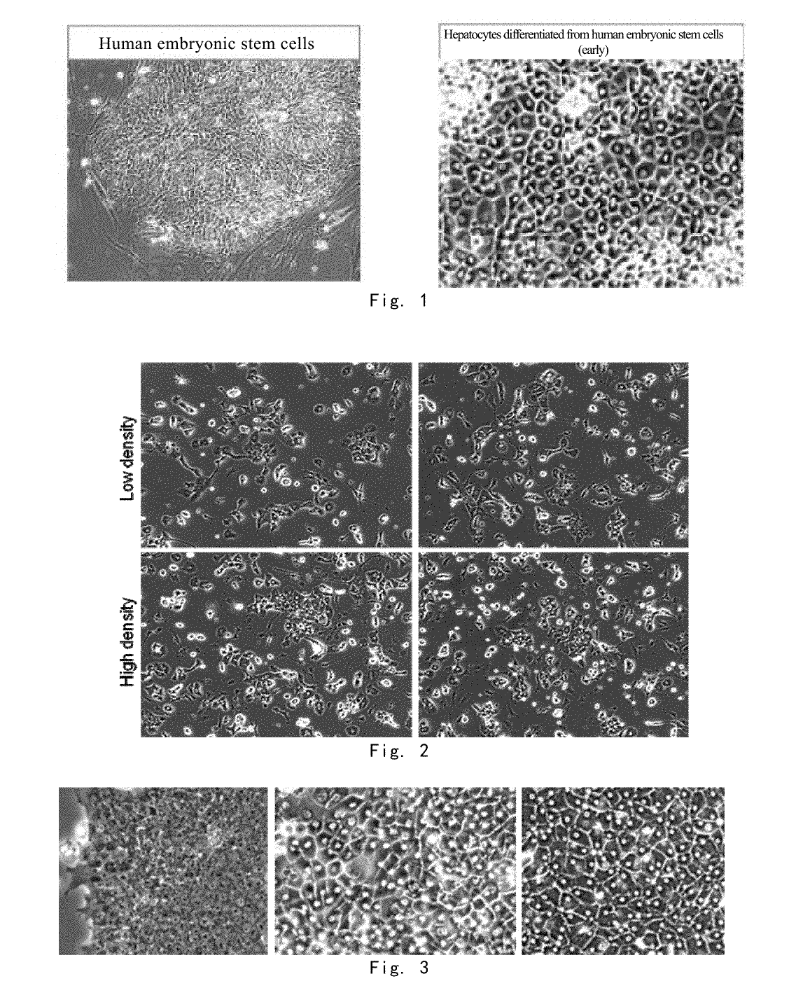

The subculturing steps were as follows: digesting human ES cells during differentiation into single cells with a digestion solution comprising trypsin, and resuspending and subculturing the single cells at a ratio of 1:2-1:5; and subculturing the differentiated cells according to the method described in “1” above. See FIG. 2 for subculture results.

### 3. Maturation Culture of Human Liver Precursor Cells

Human liver precursor cells cultured or subcultured according to the method described in 1 or 2 above were subjected to induced differentiation culture for maturation as follows: subjecting human liver precursor cells to maturation culture in “hepatocyte differentiation-strengthening medium 2” at 37° C. and 5% CO2 for 7-15 days to obtain functional human liver mature cells.

FIG. 3 shows comparison of morphology of human ES cells with differentiated liver mature cells and human primary hepatocytes, in which left panel: ES cells; middle panel: differentiated liver mature cells; right panel: primary hepatocytes.

Functional human liver mature cells obtained by maturation culture for 7-15 days can be used for cryopreservation, resuscitation, passage for a limited number of times and maintenance culture for a longer period of time, and used for various functional tests.

### Example 3. Flow Cytometric Analysis of Hepatocyte-Specific Marker Staining of Liver Mature Cells Differentiated from Human ES Cells Induced in Hepatocyte Differentiation Medium 4

The method for inducing differentiation of human ES cells into hepatocytes was the same as that of Example 2. The difference was that the cells were cultured using hepatocyte differentiation medium 4 all the time.

Liver mature cells differentiated from human ES cells were subjected to flow cytometric analysis of hepatocyte-specific marker staining Using a conventional immunostaining method, liver mature cells obtained after differentiation of human ES cells induced by the above experimental procedure were subjected to immunostaining for hepatocyte-specific markers (AAT, ALB, Asgpr, CYP3A and HNF4a). The immunostaining method was as follows:

(1) discarding the cell medium and rinsing the cells with PBS once;

(2) digesting the cells with 0.05% trypsin at 37° C. for 5 minutes, terminating the action of trypsin with a trypsin terminator or a serum/albumin-containing cell medium, centrifuging the solution at 800-1000 rpm for 3-5 minutes, and discarding the supernatant;

(3) fixing the pellet with 2% paraformaldehyde for 10 minutes, and then rinsing the pellet with PBS for 5 minutes (x3);

(4) blocking the cells with 10% sheep serum at room temperature for 60 minutes;

(5) treating the cells with 0.1% Triton for 5-10 min;

(6) adding a primary antibody (rabbit anti-ALB antibody, murine anti-CYP3A antibody or rabbit anti-Asgpr antibody), and incubating the mixture at room temperature for 1 hour or at 4° C. overnight;

(7) rinsing the mixture with PBS for 5 minutes (×3);

(8) adding a secondary antibody (Cy3-labeled goat anti-rabbit antibody, FITC-labeled goat anti-mouse or goat anti-rabbit antibody), and incubating the mixture at room temperature for 45-60 min; and

(9) washing the mixture with PBS (5 min×3).

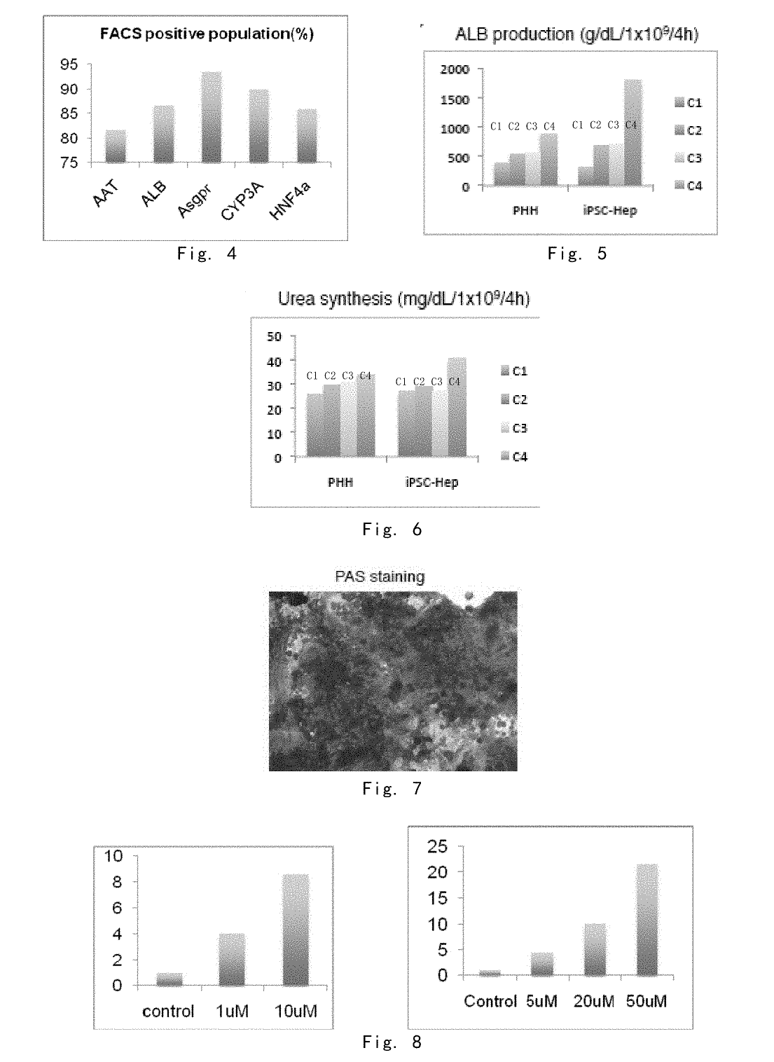

Flow cytometric analysis was then performed and the results are shown in FIG. 4.

As can be seen from FIG. 4, there is a very high proportion of differentiated liver mature cells obtained that are positive for the hepatocyte-specific markers. Therefore, it can be confirmed that functional human liver mature cells were obtained using the medium and culturing method of the present invention.

### Example 4. 4 Groups of Comparisons of Albumin Production of Liver Mature Cells Differentiated from Human iPS Cells with Primary Hepatocytes in Hepatocyte Differentiation Media 1 and 2 as Well as Hepatocyte Differentiation-Strengthening Media 1 and 4 (Corresponding to C1-C4 in Order, Respectively) Vs Hepatocyte Differentiation-Strengthening Medium 2, Respectively

The method for inducing differentiation of human iPS cells into liver mature cells was the same as that of Example 2, only except that 4 groups of different differentiation media were grouped and used simultaneously for differentiation culture and induction culture of human iPS cells.

4 groups of obtained liver mature cells differentiated from human iPS cells through induction were compared with human primary hepatocytes for albumin production. The specific method was as follows:

testing 4 groups of liver mature cells differentiated from human iPS cells through induction obtained in the above experimental procedure and commercially available human primary hepatocytes for albumin secretion using an ELISA kit; and referring to the kit instructions (Bioassay System Inc./DIAG-250, USA, BCG Albumin assay kit) for the test implementation procedure.

FIG. 5 shows the comparison results of albumin production of 4 groups of obtained liver mature cells differentiated from human iPS cells with human primary hepatocytes. As can be seen from the results, the obtained liver mature cells differentiated from human iPS cells have the function of albumin production unique to human hepatocytes.

### Example 5. 4 Groups of Comparisons of Urea Production of Liver Mature Cells Differentiated from Human iPS Cells with Primary Hepatocytes in Hepatocyte Differentiation Media 1 and 2 as Well as Hepatocyte Differentiation-Strengthening Media 1 and 4 (Corresponding to C1-C4 in Order, Respectively) Vs Hepatocyte Differentiation Medium 3, Respectively

The method for inducing differentiation of human iPS cells into liver mature cells was the same as that of Example 2, only except that 4 groups of different differentiation media were grouped and used simultaneously for differentiation culture and induced differentiation of human iPS cells.

4 groups of obtained liver mature cells differentiated from human iPS cells through induction were compared with primary hepatocytes for urea production. The specific method was as follows:

testing 4 groups of liver mature cells differentiated from human iPS cells obtained in the above experiment and commercially available human primary hepatocytes for urea synthesis using a urea nitrogen test kit; and referring to the kit instructions (Bioassay System Inc./DIUR-500, USA, Urea assay kit) for the test implementation procedure.

Urea is derived from blood ammonia in the blood, and its production reflects the detoxification ability of hepatocytes. FIG. 6 shows the comparison results of urea production of 4 groups of hepatocytes differentiated from human iPS cells with human primary hepatocytes.

As can be seen from FIG. 6, the obtained liver mature cells differentiated from human iPS cells have the function of urea production unique to hepatocytes.

### Example 6. Glycogen Staining of Liver Mature Cells Differentiated from Human ES Cells Induced in Hepatocyte Differentiation Medium 5 and Hepatocyte Differentiation-Strengthening Medium 3

The method for inducing differentiation of human ES cells into liver mature cells was the same as that of Example 2, only except that hepatocyte differentiation medium 5 was used for differentiation culture and hepatocyte differentiation-strengthening medium 3 was used for differentiation and maturation culture.

Liver mature cells differentiated from human ES cells were subjected to glycogen staining. The degree of staining reflects the ability of hepatocytes to store glycogen. Liver glycogen staining was performed using the Schiff method. The specific method was as follows:

(1) discarding the cell medium and rinsing the cells with PBS once;

(2) fixing the cells with 4% paraformaldehyde for 10 minutes, and then rinsing same with PBS for 5 minutes (x3);

(3) adding the PAS-I solution and keeping for 10 minutes and rinsing same with running water;

(4) adding the PAS-II solution and keeping for 1-2 min and rinsing same with running water; and

(5) taking photos with a microscope.

FIG. 7 shows results of glycogen staining of liver mature cells differentiated from human ES cells. The cultured cells were positive for liver glycogen staining. As can be seen from the results, the liver mature cells obtained by the method of the present invention have the same glycogen storage activity as human hepatocytes.

### Example 7. Activity Induction of P450 Enzymes (CYP3A4 and CYP1A2) of Liver Mature Cells Differentiated from Human ES Cells Induced in Hepatocyte Differentiation-Strengthening Medium 4 and Hepatocyte Differentiation-Strengthening Medium 2

The method for inducing differentiation of human ES cells into liver mature cells was the same as that of Example 2, only except that hepatocyte differentiation-strengthening medium 4 was used for differentiation culture and hepatocyte differentiation-strengthening medium 2 was used for continuous differentiation and maturation culture.

The activities of P450 enzymes (CYP3A4 and CYP1A2) in liver mature cells differentiated from human ES cells was inducted:

(1) The activity of P450 enzyme (CYP3A4) in liver mature cells differentiated from human ES cells induced in the above experimental procedure was enhanced when induced by rifampicin. The method of activity induction of P450 enzyme was as follows:

The liver mature cells were treated with different concentrations (1 uM, 10 uM and 25 uM) of rifampicin, and a treatment group without rifampicin and with the same conditions was used as the control. Liver mature cells differentiated from human ES cells induced in each treatment group were tested for the P450 enzyme (CYP3A4) activity using PromegaP450-Glo™ Assays kit; and referring to the kit instructions (Promega Inc., USA) for the implementation procedure.

(2) The activity of P450 enzyme (CYP1A2) in liver mature cells differentiated from human ES cells induced in the above experimental procedure was enhanced when induced by omeprazole. The method of activity induction of P450 enzyme was as follows:

The liver mature cells were treated with different concentrations (1 uM, 10 uM and 25 uM) of omeprazole, and a treatment group without omeprazole and with the same conditions was used as the control. Liver mature cells differentiated from human ES cells induced in each treatment group were tested for CYP1A2 gene expression quantification using qRT-PCR.

FIG. 8 shows results of activity induction of P450 enzymes (CYP3A4 and CYP1A2), in which left panel: the activity of CYP3A4 is enhanced when induced by different concentrations of rifampicin; right panel: the activity of CYP1A2 is enhanced when induced by different concentrations of omeprazole. As can be seen from FIG. 8, the mature hepatocytes obtained by the method of the present invention have high activity of P450 metabolic enzymes.

### Example 8. Comparison of Morphology of Liver Precursor Cells Directly Differentiated from Human ES Cells with Hepatocytes Induced in Hepatocyte Differentiation Medium 6

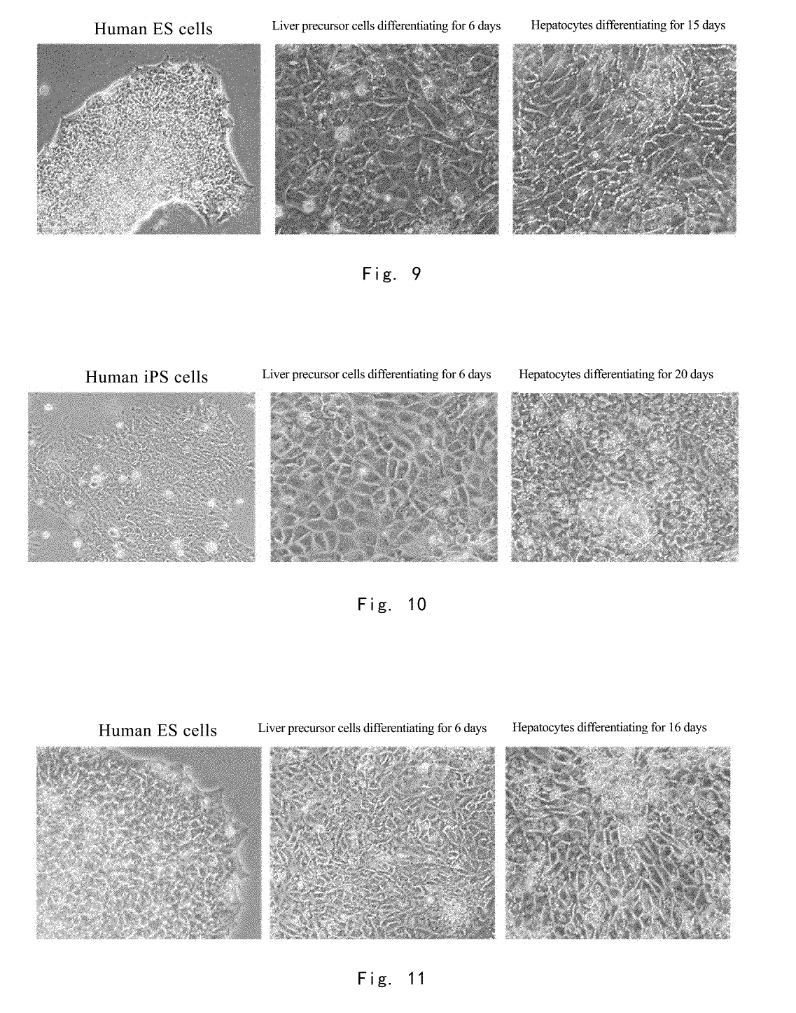

The methods for inducing differentiation of human ES cells into liver precursor cells and liver (mature) cells were the same as that of Example 3; and the results are shown in FIG. 9, which left panel: human ES cells; middle panel: differentiated liver precursor cells; right panel: differentiated hepatocytes.

FIG. 9 shows that the combination of small molecules comprising GSK3β inhibitor BIO, a TGFβ inhibitor and retinoic acid can also induce direct differentiation of stem cells into liver precursor cells and liver mature cells.

### Example 9. Comparison of Morphology of Liver Precursor Cells Directly Differentiated from Human iPS Cells with Hepatocytes Induced in Hepatocyte Differentiation Medium 7

The methods for inducing differentiation of human iPS cells into liver precursor cells and liver (mature) cells were the same as that of Example 3; and the results are shown in FIG. 10, in which left panel: human iPS cells; middle panel: differentiated liver precursor cells; right panel: differentiated hepatocytes.

FIG. 10 shows that the combination of small molecules comprising TGFβ inhibitor RepSox, a GSK3β inhibitor and retinoic acid can also induce direct differentiation of stem cells into liver precursor cells and liver mature cells.

### Example 10. Comparison of Morphology of Liver Precursor Cells Directly Differentiated from Human ES Cells with Hepatocytes Induced in Hepatocyte Differentiation Medium 8

The methods for inducing differentiation of human ES cells into liver precursor cells and liver (mature) cells were the same as that of Example 3; and the results are shown in FIG. 11, in which left panel: human ES cells; middle panel: differentiated liver precursor cells; right panel: differentiated hepatocytes.

FIG. 11 shows that the combination of small molecules comprising GSK3β inhibitor CHIR-98014, TGFβ inhibitor SB525334 and retinoic acid can also induce direct differentiation of stem cells into liver precursor cells and liver mature cells.

In summary, the novel method of the present invention for inducing direct differentiation of human stem cells (such as embryonic stem cells or induced pluripotent stem cells) into hepatocytes has the following features:

1. Only combinations of small molecules are used to multi-target-directed induce direct differentiation of human ES cells or iPS cells into hepatocytes. No stepwise induction is needed, and various cell growth factors are not used. Since small molecules are stable in nature, differentiation results are stable and safe, and the cost is greatly reduced.

2. The differentiation stage is clear and the quality is easy to control.

3. The direct differentiation efficiency is high, that is to say, 1×10⁶ human ES/iPS cells can differentiate into 5-10×10⁶ or more functional hepatocytes.

4. The directly differentiated hepatocytes have high purity without the need for additional purification means (Asgp≥85%), thus the method does not impair the cell activity, is simple to operate and have reduced cost.

5. The directly differentiated hepatocytes have complete functions, such as albumin production, urea synthesis, glycogen storage, activity induction of P450 enzymes and other abilities.

6. The direct differentiation method is well universal and reproducible, in which 2 human ES cell lines and 7 human iPS cell lines are all differentiated into functional liver mature cells.

7. The differentiated liver precursor cells can be used for proliferation, passage, cryopreservation and resuscitation, and can further differentiate into functional liver mature cells; and the obtained liver mature cells can be used for cryopreservation, resuscitation, limited passage and maintenance culture.

8. The differentiated hepatocytes are highly consistent with human primary hepatocytes in morphology and function.

9. No animal feeder cells are used to culture ES/iPS cells, thus there is no phenomenon of contamination with animal-derived materials.

10. GSK3β inhibitors and TGFβ inhibitors comprise a series of small molecules, which can be divided into two types but have the same function or induce the same target, and different combinations formed thereby can all induce stem cells to differentiate directly into hepatocytes to varying degrees.

11. The method is simple and easy to operate; and there is no need to form embryo bodies (EBs).

12. The method has the features of conventional culturing, short cycle and suitable for mass production and easy industrialization and the like.

All references mentioned in the present invention are incorporated by reference in this application, as if each reference were individually incorporated by reference. In addition, it should be understood that after reading the above teachings of the present invention, those skilled in the art can make various changes or modifications to the present invention, and these equivalent forms also fall within the scope defined by the appended claims of the present application.

1. A medium for inducing directed differentiation of human stem cells into hepatocytes comprising a cell differentiation minimal medium; and
a GSK3β inhibitor with a final concentration of 0.5-8 uM;
a TGFβ inhibitor with a final concentration of 0.1-10 uM; and
a retinoid with a final concentration of 0.001-10 uM;
wherein the medium can induce directed differentiation of human stem cells into hepatocytes directly, thereby obtaining human liver precursor cells or liver mature cells.

2. The medium according to claim 1, wherein,
the GSK3β inhibitor is present at a final concentration of 0.5-5 uM;
the TGFβ inhibitor is present at a final concentration of 0.5-8 uM; and
the retinoid is present at a final concentration of 0.01-5 uM.

3. The medium according to claim 1, wherein the GSK3β inhibitor is selected from GSK3β signaling pathway inhibitors or compounds of the same type that have the same function or induce the same target and are represented by CHIR-99021, BIO, AZD2858, TWS119, CHIR-98014, etc., or combinations thereof;
the TGFβ inhibitor is selected from TGFβ signaling pathway inhibitors or compounds of the same type that have the same function or induce the same target and are represented by SB431542, A83-01, SB525334, LY2109761, RepSox, etc., or combinations thereof;
the retinoid is natural or synthetic and includes retinoid differentiation agents or compounds of the same type that have the same function or induce the same target and are represented by retinoic acid (alternative name: all trans retinoic acid), 13-cis retinoic acid, 9-cis-retinoic acid, etc., or combinations thereof; and
the Rock inhibitor is selected from Rock signaling pathway inhibitors or compounds of the same type that have the same function or induce the same target and are represented by Y-27632 (alternative name: Y-27632 2HCl), GSK429286A, RKI-1447, etc, or combinations thereof.

4. The medium according to claim 3, wherein the GSK3β inhibitor is GSK3β inhibitor CHIR-99021; or
the TGFβ inhibitor is TGFβ inhibitor SB431542 or/and A83-01; or
the retinoid is retinoic acid; or
the Rock inhibitor is Rock inhibitor Y-27632.

5. The medium according to claim 1, wherein
the GSK3β inhibitor is CHIR-99021 or/and CHIR-98014 with a final concentration of 0.5-8 uM;
the TGFβ inhibitor is SB431542 or/and A83-01 or/and RepSox with a final concentration of 0.1-10 uM; and
the retinoid is retinoic acid with a final concentration of 0.001-10 uM.

6. The medium according to claim 5, wherein
the GSK3β inhibitor is CHIR-99021 or/and CHIR-98014 with a final concentration of 0.5-5 uM;
the TGFβ inhibitor is SB431542 or/and A83-01 or/and RepSox with a final concentration of 0.5-8 uM; and
the retinoid is retinoic acid with a final concentration of 0.01-5 uM.

7. The medium according to claim 1, further comprising:
Rock inhibitor with a final concentration of 0.5-50 uM; and/or
hepatocyte growth factor with a final concentration of 5-100 ng/ml; and/or
oncostatin M with a final concentration of 1-100 ng/ml; and/or
dexamethasone with a final concentration of 0.5-20 uM;
wherein the medium to which the above components are added can increase the cell survival rate or promote the differentiation and maturation of ES/iPS cells into hepatocytes and maintain the growth of liver mature cells.

8. The medium according to claim 7, comprising:
Rock inhibitor with a final concentration of 1-20 uM; and/or
hepatocyte growth factor with a final concentration of 5-40 ng/ml; and/or
oncostatin M with a final concentration of 5-50 ng/ml; and/or
dexamethasone with a final concentration of 2.5-10 uM.

9. (canceled)

10. (canceled)

11. A kit for inducing directed differentiation of human stem cells into hepatocytes directly, characterized in that the kit comprises a GSK3β inhibitor, a TGFβ inhibitor and a retinoid.

12. The kit according to claim 11, further comprising a Rock inhibitor, hepatocyte growth factor, oncostatin M or dexamethasone.

13. A method for inducing directed differentiation of human stem cells into hepatocytes directly, comprising combining human stem cells with the medium according to of claim 1 to induce directed differentiation of the human stem cells into hepatocytes directly.

14. The method according to claim 13, further comprising:
(1) coating a culture plate with one of matrigel, rat tail gel, gelatin, fibronectin and vitronectin for 30 minutes to 24 hours; then suspending human stem cells in the medium and plating the suspension in the culture plate; and culturing the culture plate at 37° C.±1° C. and 5% CO₂, with the medium changed every 72 hours, so as to initiate human liver precursor cell differentiation;
(2) subculturing the cells at a ratio of 1:2-1:5 if the confluence reaches 90%, wherein subculturing comprises
digesting the differentiated human stem cells into single cells with a digestion solution comprising trypsin, EDTA, Acutase, or Tryple E, and resuspending and subculturing the single cells at a ratio of 1:2-1:5 at 37° C.±1° C. and 5% CO₂, with the medium changed every 72 hours;
(3) differentiating to obtain human liver precursor cells comprising performing differentiation culture of the cells for 10-15 days according to the method described in steps (1) and (2) to obtain human liver precursor cells which can be used for cryopreservation, resuscitation and passage and can be further induced to differentiate into functional human liver mature cells; and
(4) maturing the culture of human liver precursor cells comprising performing further differentiation culture of the human liver precursor cells obtained by the differentiation culture in step (3) in the medium for 7-15 days at 37° C.±1° C. and 5% CO₂ to obtain functional human liver mature cells which can be used for cryopreservation, resuscitation and limited passage.

15. The medium according to claim 1 wherein the human stem cells comprise human embryonic stem cells, induced pluripotent stem cells, mesenchymal stem cells, adipose stem cells, umbilical cord blood stem cells or human stem cells having multi-lineage differentiation potential.

16. The method of claim 13 wherein
GSK3β inhibitor is selected from GSK3β signaling pathway inhibitors or compounds of the same type that have the same function or induce the same target and are represented by CHIR-99021, BIO, AZD2858, TWS119, CHIR-98014, etc., or combinations thereof;
the TGFβ inhibitor is selected from TGFβ signaling pathway inhibitors or compounds of the same type that have the same function or induce the same target and are represented by SB431542, A83-01, SB525334, LY2109761, RepSox, etc., or combinations thereof;
the retinoid is natural or synthetic and includes retinoid differentiation agents or compounds of the same type that have the same function or induce the same target and are represented by retinoic acid (alternative name: all trans retinoic acid), 13-cis retinoic acid, 9-cis-retinoic acid, etc., or combinations thereof.

17. The kit of claim 11 wherein the human stem cells comprise human embryonic stem cells, induced pluripotent stem cells, mesenchymal stem cells, adipose stem cells, umbilical cord blood stem cells or human stem cells having multi-lineage differentiation potential.

18. The method of claim 13 wherein the human stem cells comprise human embryonic stem cells, induced pluripotent stem cells, mesenchymal stem cells, adipose stem cells, umbilical cord blood stem cells or human stem cells having multi-lineage differentiation potential.
